(19) United States
(12) Patent Application Publication
(10) Pub. No.: US 2001/0037077 A1
(43) Pub. Date: Nov. 1, 2001

(54) Super care bandages

(76) Inventor: Brian James Wiemken, Temecula, CA (US)

Correspondence Address: Matthew J. Peirce, Esq., 1550 Starlight Canyon Avenue, Las Vegas, NV, 89123, US

(21) Appl. No.: 09/833,587

(22) Filed: Apr. 13, 2001

Related U.S. Application Data
(60) Provisional application No. 60/202,192, filed on May 5, 2000.

(51) Int. Cl. A61F 13/00

(52) U.S. Cl. 602/41

(57) ABSTRACT

A new and improved apparatus is disclosed for covering the sores, scrapes, and cuts of children and adults. The present invention would be a new and improved bandage which would come in two different embodiments, a children's version embodiment and an adult version embodiment. Both embodiments would have two ends, a first end and a second end, and would also have two sides, a sticky side and a non-sticky side. A pad would be attached to the sticky side of each embodiment closer to the first end of the bandage than the second end of the bandage. The much longer sticky portion of the bandage near the second end would allow the present invention, once placed over a cut or sore on a finger, to have the second end of the bandage overlap a significant portion of the first end of the bandage, creating a stronger bond between the two ends of the bandage.

### I. BACKGROUND OF THE INVENTION

The present invention concerns that of a new and improved apparatus for covering the sores, scrapes, and cuts of children and adults.

### II. DESCRIPTION OF THE PRIOR ART

U.S. Pat. No. 5,843,018, issued to Shesol et al., discloses a disposable sterile emollient carrier device for delivering an application of an emollient.

U.S. Pat. No. 5,267,952, issued to Gardner, discloses an elongate bandage that is predominantly stretchable in the longitudinal direction which enables readily applied development of an elastic surgical limb sleeve that can be rendered locally inextensible.

U.S. Pat. No. 2,068,703, issued to Powdermaker, discloses a surgical bandage which is adapted to be wrapped about a body member to be dressed and wherein contact of an adhesive with the skin is completely eliminated.

### III. SUMMARY OF THE INVENTION

The present invention concerns that of a new and improved apparatus for covering the sores, scrapes, and cuts of children and adults. The present invention would be a new and improved self-adhering bandage which would come in two different embodiments, a children's version embodiment and an adult version embodiment. The children's version embodiment would be analogous to a standard bandage, having a first end, a second, and a pad attached in the middle. However, the first end of the bandage would be shortened because a child's finger is smaller in diameter than that of an adult. The adult version embodiment would also be analogous to a standard bandage, having a first end, a second, and a pad attached in the middle, but instead would have a second end that would be significantly longer than that of a standard bandage. The longer second end of the bandage increases the adhesion contact area, thereby allowing it to overlap back on itself to enhance adhesion and not detach itself so easily.

There has thus been outlined, rather broadly, the more important features of a self-adhering bandage in order that the detailed description thereof that follows may be better understood and in order that the present contribution to the art may be better appreciated. There are, of course, additional features of the self-adhering bandage that will be described hereinafter and which will form the subject matter of the claims appended hereto.

In this respect, before explaining at least one embodiment of the self-adhering bandage in detail, it is to be understood that the self-adhering bandage is not limited in its application to the details of construction and to the arrangements of the components set forth in the following description or illustrated in the drawings. The self-adhering bandage is capable of other embodiments and being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present self-adhering bandage. It is important, therefore, that the claims be regard as including such equivalent constructions insofar as they do not depart from the spirit and scope of the present invention.

It is therefore an object of the present invention to provide a self-adhering bandage which has all of the advantages of the prior art and none of the disadvantages.

It is another object of the present invention to provide a self-adhering bandage which may be easily and efficiently manufactured and marketed.

It is another object of the present invention to provide a self-adhering bandage which is of durable and reliable construction.

Other objects, features and advantages of the present invention will become more readily apparent from the following detailed description of the preferred embodiment when considered with the attached drawings and appended claims.

### IV. BRIEF DESCRIPTION OF THE DRAWINGS

FIG. 1 shows a top view of the children's version embodiment of the present invention.

FIG. 2 shows a top view of the adult version embodiment of the present invention.

FIG. 3 shows a perspective view of the adult version embodiment of the present invention in use.

### V. DESCRIPTION OF THE PREFERRED EMBODIMENT

FIG. 1 shows a top view of the children's version embodiment 1 of the present invention. Children's version embodiment 1 includes bandage 2, which has a first end 4 and a second end 6. Bandage 2 also has two sides, a sticky side 5 and a non-sticky side 7. Bandage 2 has pad 8, which is firmly attached to sticky side 5 slightly off the halfway point between the first end 4 and the second end 6.

Because pad 8 would be attached to the sticky side 5 of bandage 2 slightly off-center, the sticky area of sticky side 5 near the first end 4 of bandage 2 would be shorter than the sticky area on sticky side 5 near the second end 6 of bandage 2. This characteristic is preferred in the children's version embodiment 1 so that this embodiment would work better with a children's finger, which is usually significantly smaller than that of an adult's finger.

In use, a child would place the pad 8 against a sore or cut and wrap the sticky area of sticky side 5 near the first end 4 of bandage 2 around a finger until the first end 4 of bandage 2 is placed into contact with the pertinent finger. Then, the user would grasp the sticky area of sticky side 5 near the second end 6 of bandage 2 and proceed to wrap it around the pertinent finger so as to allow the sticky side 5 of bandage 2 to be placed flush against the finger. A user would wrap the second end 6 of bandage 2 around the pertinent finger until the second end 6 of bandage 2 is placed into contact with either the pertinent finger or the second side of bandage 2, which would be already attached to the finger.

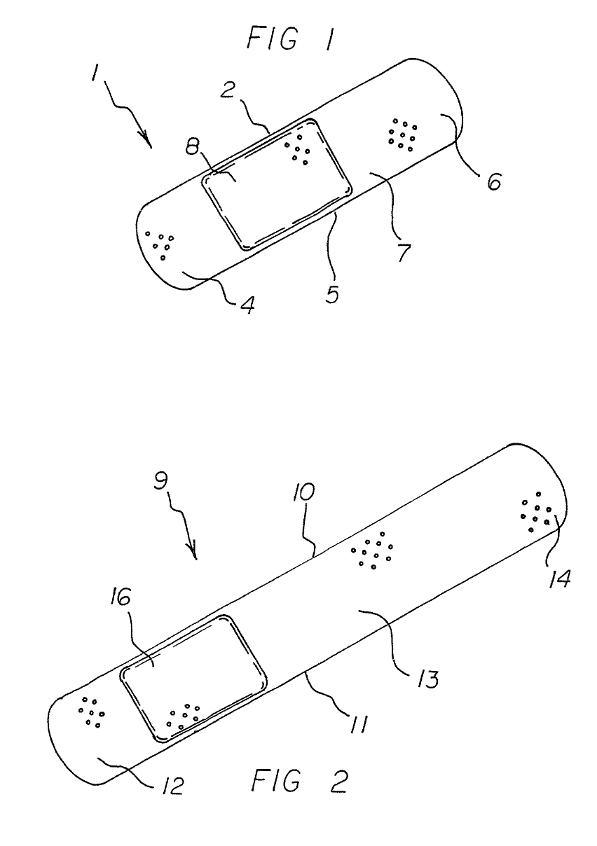

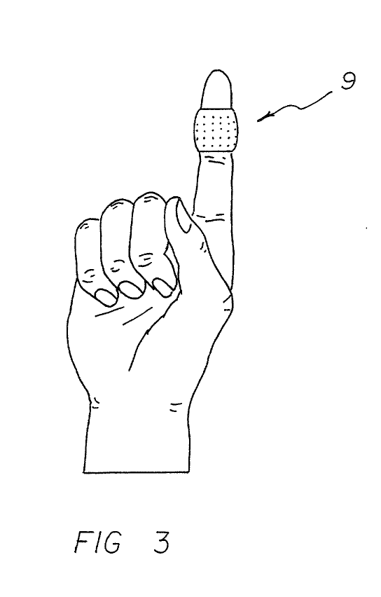

FIG. 2 shows a top view of the adult version embodiment 9 of the present invention. Adult version embodiment 9 includes bandage 10, which has a first end 12 and a second end 14. Bandage 10 also has two sides, a sticky side 11 and a non-sticky side 13 Bandage 10 has pad 16, which is firmly attached to sticky side 11 significantly closer to the first end 12 than the second end 14.

Because pad 16 would be attached to the sticky side 11 of bandage 10 significantly closer to the first end 12 than the second end 14 of bandage 10, the sticky area of sticky side 11 near the first end 12 of bandage 10 would be a lot shorter than the sticky area on sticky side 11 near the second end 14 of bandage 10. This characteristic is preferred in the adult version embodiment 9 so that this embodiment would work better with an adult's finger, which is usually significantly larger than that of a child's finger.

In use, an adult would place the pad 16 against a sore or cut and wrap the sticky area of sticky side 11 near the first end 12 of bandage 10 around a finger until the first end 12 of bandage 10 is placed into contact with the pertinent finger. Then, the user would grasp the sticky area of sticky side 11 near the second end 14 of bandage 10 and proceed to wrap it around the pertinent finger so as to allow the sticky side 11 of bandage 10 to be placed flush against the finger. A user would wrap the second end 14 of bandage 10 around the pertinent finger until the second end 14 of bandage 10 is placed into contact with either the pertinent finger or the second side of bandage 10, which would be already attached to the finger.

FIG. 3 shows a perspective view of the adult version embodiment 9 of the present invention in use. Adult version embodiment 9 is wrapped around an adult's finger to the point where the second end 14 is significantly overlapping the first end 12 of adult version embodiment 9.

With both embodiments of the present invention, allowing the second end of the bandage to overlap the first end of the bandage allows a significant portion of the sticky side of the respective bandage to attach to the bandage instead of directly to the skin itself By having this characteristic, the present invention increases the chance that a bandage will stay on a user's finger until a user actually chooses to take it off. Presently, bandages typically fall off or become loose after a user has gotten them wet, either through sweating, swimming, or related activity. Even if a second end of a bandage overlaps the first end of a bandage slightly, the degree of overlap is not enough to cause significant bonding action between the two ends of the bandage. The present invention alleviates that concern by providing significant overlap and holding power for a bandage on a user's finger.

What I claim as my invention is:

1. A bandage comprising
(a) two sides, a first side and a second side, the first side of the bandage having a attached adhesive uniformly applied to the first side of the bandage,
(b) two ends, a first end and a second end, and
(c) a pad attached to the first side of the bandage, the pad being attached slightly closer to the first end of the bandage than the second end of the bandage.

2. A bandage comprising
(a) two sides, a first side and a second side, the first side of the bandage having a attached adhesive uniformly applied to the first side of the bandage,
(b) two ends, a first end and a second end, and
(c) a pad attached to the first side of the bandage, the pad being attached significantly closer to the first end of the bandage than the second end of the bandage.
